(19) United States
(12) Patent Application Publication
(10) Pub. No.: US 2024/0139092 A1
(43) Pub. Date: May 2, 2024

(54) ANHYDROUS COSMETIC CONCEALER COMPOSITIONS HAVING LOW FILLER LEVELS

(71) Applicant: L'OREAL, PARIS (FR)

(72) Inventors: Francesca Thuy-Trang CAO, Piscataway, NJ (US); Sarah Kathryn YURO, Jackson, NJ (US)

(73) Assignee: L'OREAL, PARIS (FR)

(21) Appl. No.: 17/975,284

(22) Filed: Oct. 27, 2022

(51) Int. Cl. A61K 8/92; A61K 8/25; A61K 8/37; A61K 8/81; A61Q 1/02; A61Q 19/00

(52) U.S. Cl. CPC A61K 8/92; A61K 8/25; A61K 8/37; A61K 8/8111; A61Q 1/02; A61Q 19/00; A61K 2800/31; A61K 2800/43; A61K 2800/805

(57) ABSTRACT

Cosmetic concealer compositions including at least about 25% by weight of one or more oils are provided. The compositions include from 0.5% by weight to about 4% by weight of one or more fillers, wherein the one or more fillers comprise silica aerogel, borosilicate, swellable clay, and combinations thereof. The compositions further include at least 25% by weight of one or more pigments. The compositions are substantially free of water.

### FIELD OF THE INVENTION

The present invention relates to cosmetic concealer compositions comprising low concentrations of fillers.

### DISCUSSION OF THE BACKGROUND

Pigmented cosmetic compositions designed to conceal skin imperfections are known. Such compositions may take various forms, such liquids, creams, loose powders, pressed powders, hot pour compositions, sticks, etc. However, these compositions have one or multiple drawbacks in various properties.

The inventors of the instant invention have recognized that many of these prior art compositions do not simultaneously possess a combination of a properties that would be very advantageous for “spot treatment” of skin imperfections. For example, the present inventors have recognized that it would be desirable to have a composition that provides good tack and skin adhesion yet also has a pleasant gliding sensation when applied. The present inventors have separately and further recognized that it would be desirable for such a composition to be used with various levels and types of pigments to allow for concealing performance for a wide range of skin tones, particularly darker skin tones.

Accordingly, certain aspects of the present invention relate to a makeup and/or treatment composition for keratinous materials which can conceal skin imperfections, tolerate formulation with darker pigments and/or high pigment loads and have surprisingly one or more cosmetic properties such as, for example, good tack and skin adhesion, a pleasant sensation upon application, an appropriate level of shine, and/or convenient application to the skin.

### SUMMARY OF THE INVENTION

The present invention relates to compositions useful as cosmetic concealers, that include at least about 25% by weight of one or more oils. The compositions further include from 0.5% by weight to about 4% by weight of at least one filler, wherein the at least one filler comprises silica aerogel, borosilicate, swellable clay, and combinations thereof. The compositions further include at least 25% by weight of at least one pigment, wherein the composition is substantially free of water.

According to certain aspects, compositions of the present invention include (1) from about 25% to about 70% by weight of one or more lipids, wherein the one or more lipids include (a) at least one oil, (b) a high melting point hydroxylated fatty acid ester; (c) at least one wax; and (d) at least one paste-like compound; (2) one or more inorganic particulates, wherein the one or more inorganic particulates include (a) at least one filler, present in a concentration by weight of from about 0.5% by weight to about 4% with respect to the entire composition, wherein the at least one at least one filler is selected from a group consisting of silica aerogel, borosilicate, swellable clay, and combinations thereof; (b) one or more pigments, present in a concentration by weight of at least about 25% by weight with respect to the entire composition; wherein the at least one pigment includes a pigment selected color pigments, titanium dioxide, and combinations thereof; and (3) from about 10% by weight to about 30% by weight of an unsaturated aliphatic tackifier having a weight average molecular weight of less than about 10,000 daltons; wherein the composition is substantially free of water, boron nitride, talc, mica, bismuth oxychloride (“BiOCl”), and perlite. The paste-like compound may be in a concentration by weight from about 5% to about 15% of the entire composition. Color pigments (e.g., iron oxides) may be included in the composition in concentrations by weight of at least about 5%, such as at least about 10%, such as from about 10% to about 30%.

According to another aspect of the invention, a method of making up a keratinous substrate includes a method of concealing imperfections on a keratinous substrate comprising topically applying the above-mentioned compositions to selected imperfections on the keratinous substrate.

It is to be understood that both the foregoing general description and the following detailed description are exemplary and explanatory only, and are not restrictive of the invention.

### DETAILED DESCRIPTION OF THE INVENTION

As used herein, the expression “at least one” means one or more and thus includes individual components as well as mixtures/combinations.

As used herein, all ranges provided are meant to include every specific range within, and combination of sub ranges between, the given ranges. Furthermore, notably the range description of the type “from about 1%, 2% or 3% to about 5%, 10% or 15%,” includes about 1% to about 5%, about 1% to about 10%, about 1% to about 15%, about 2% to about 5%, about 2% to about 10%, about 2% to about 15%, about 3% to about 5%, about 3% to about 10%, and/or about 3% to about 15%. Other than in the operating examples, or where otherwise indicated, all numbers expressing quantities of ingredients and/or reaction conditions are to be understood as being modified in all instances by the term “about,” meaning within 10% to 15% of the indicated number. So “about 10%” means from 8.5% to 11.5%, such as from 9% to 11%.

“Actives basis” as used herein means considering only the particular component of ingredient (e.g., in a composition) and ignoring other chemically unrelated components that may also be present in the same raw material source of that particular component.

“Film former” or “film forming agent” as used herein means any material such as, for example, a polymer or a resin that leaves a film on the substrate to which it is applied.

“Polymer” as used herein means a compound which is made up of at least two monomers.

“Keratinous materials” includes materials containing keratin such as hair, skin, eyebrows, lips and nails.

“Solids basis” as used herein means considering only components (e.g., in a composition) that are solid at room temperature and ignoring portions of the composition that are liquid, e.g., water and other volatile solvents.

“Substituted” as used herein, means comprising at least one substituent. Non-limiting examples of substituents include atoms, such as oxygen atoms and nitrogen atoms, as well as functional groups, such as hydroxyl groups, ether groups, alkoxy groups, acyloxyalky groups, oxyalkylene groups, polyoxyalkylene groups, carboxylic acid groups, amine groups, acylamino groups, amide groups, halogen containing groups, ester groups, thiol groups, sulphonate groups, thiosulphate groups, siloxane groups, hydroxyalkyl groups, and polysiloxane groups. The substituent(s) may be further substituted.

“Volatile”, as used herein, means having a flash point of less than about 100° C.

“Non-volatile”, as used herein, means having a flash point of greater than about 100° C.

“Substantially free” means having less than 1% by weight of a particular component, preferably less than 0.5% by weight, such as less than 0.25% by weight, such as less than 0.1% by weight.

“Anhydrous” means the compositions are substantially free of water, and preferably have no water.

All percentages of ingredients herein are listed on an actives basis and on a weight basis with respect to the entire composition unless specifically stated otherwise.

The composition of the present invention may be of various forms including semi-solid, and fluid under shear.

The composition of the invention generally include at least one oil and optionally other lipids; at least one filler, at least one pigment and optionally other inorganic particulates; and are substantially free of water. The lipid(s) may (co)exist, for example in a fatty phase, such as one having inorganic particulates dispersed therein. Optional ingredients such as aliphatic tackifiers, polymers, etc., may be dissolved or dispersed in the fatty phase. Many other optional ingredients such as other particulates, preservatives, skin active ingredients, and the like may also be dissolved or dispersed in the fatty phase.

“Inorganic particulate,” as used herein means any finely divided material that is predominantly inorganic (including inorganic particulates having an organic or silicon-based coating, but excluding silicone elastomer particulates). Inorganic particulates encompass pigments, and fillers.

Although the relative proportions of these components may vary, according to certain embodiments of the invention, the composition may include a concentration of lipids and inorganic particulates such that the total (combined) concentrations of lipids and inorganic particulates may be at least about 60% by weight of the total composition. In certain embodiments the amount of lipids and inorganic particulates are present in a concentration by weight of at least about 60% by weight of the total composition, such as at least about 70%, by weight of the total composition.

According to certain other embodiments, the lipids and inorganic particulates are present in a weight ratio of lipid to inorganic particulate that is from about 1:2 to about 2:1, such as from about 1:1 to about 2:1.

According to certain other embodiments, the composition includes from about 25% or 30% by weight to about 60% or 70% by weight of lipids and from about from about 15% or 25% by weight to about 45% or 60% by weight of inorganic particulates.

The compositions and methods of the present invention can comprise, consist of, or consist essentially of the essential elements and limitations of the invention described herein, as well as any additional or optional ingredients, components, or limitations described herein or otherwise useful in personal care.

Filler

Compositions of the present invention include certain inorganic particulates such as at least one filler. The at least one filler provides one or more benefits including opacity/hiding powder to aid in concealing skin imperfections, modification of the texture of the formula, oil absorption, as well as possible mattifying and optical/soft focus effects, but without causing undue whitening of the skin. Suitable fillers include any of various inorganic particulates that provide some opacity without generally appearing too white or whitish on the skin. Particularly suitable classes of fillers are ‘non-whitening’ fillers especially borosilicates, silica aerogels and swellable clays.

Examples of non-whitening fillers include borosilicates such as aluminum borosilicates such as calcium aluminum borosilicate. Borosilicates may be hollow borosilicate particles according to the invention are typically homogeneous and essentially uniform in sphericity. While the mean particle diameter of the hollow borosilicate particles can range up to 100 μm, the mean particle diameter is typically between 3 μm and 20 μm, and preferably between 9 μm and 13 μm.

In some embodiments, the hollow borosilicate particles comprise an alkali metal or alkaline earth metal, such as calcium and/or sodium, and may optionally contain a transition metal or post-transition metal, such as aluminum. A preferred embodiment is calcium aluminum borosilicate particles. Other suitable the hollow borosilicate particles according to the present invention include those sodium borosilicate particulates marketed by PQ Corporation under the trademark Q-CEL. Also suitable are those calcium/sodium borosilicate hollow microspheres marketed by 3M.

Preferred hollow borosilicate particles are typically high slip, chemically inert, non-absorbent, non-porous free-flowing white powders. Preferred hollow borosilicate particles according to this invention include those marketed by Presperse Inc. under the trademark LUXSIL®.

In some embodiments, the hollow borosilicate particles have densities between 1.05 g/cc and 1.15 g/cc, have a weight loss on drying of less than 0.5%, and contain, e.g., less than 20 ppm of lead and less than 3 ppm of arsenic.

Other suitable non-whitening fillers include swellable clays Compositions of the present invention include a swellable clay. By “swellable clay” it is meant a clay material that is capable of swelling in water. An example of a swellable clay are smectite clays. The crystal structure of the smectite group, is an octahedral alumina sheet between two tetrahedral silica sheets. In one notable embodiment, the swellable clay is bentonite. Bentonite is a rock formed of highly colloidal and plastic clays composed mainly of montmorillonite, a clay mineral of the smectite group, and is produced by in situ devitrification of volcanic ash. In addition to montmorillonite, bentonite may contain feldspar, cristobalite, and crystalline quartz. Bentonite has an ability to form thixotrophic gels with water, an ability to absorb large quantities of water. Variations in interstitial water and exchangeable cations in the interlayer space affect the properties of bentonite and thus the commercial uses of the different types of bentonite.

One notable swellable clay suitable for use in the composition is BENTONE GEL GTCC V, commercially available from Elementis Specialties, East Windsor, New Jersey. BENTONE GEL GTCC V is a dispersion of organically modified hectorite in caprylic/capric triglycerides with added propylene carbonate.

The concentration of the swellable clay, if present, in the composition may range from about 0.1% to about 5% by weight as from about 0.2% to about 0.65% by weight, such as from about 0.2% to about 0.45% in the composition.

Other suitable non-whitening fillers include silica aerogels. Suitable silica aerogels are hydrophobic silicas and include, but are not limited to pyrogenic silica with hydrophobic surface treatment whose particle size is less than 1 micron, preferably less than 500 nm, preferably less than 100 nm, preferably from 5 nm to 30 nm, including all ranges and subranges therebetween. It is in fact possible to modify the surface of silica chemically, by a chemical reaction producing a decrease in the number of silanol groups present on the surface of the silica. The silanol groups can notably be replaced with hydrophobic groups: a hydrophobic silica is then obtained. The hydrophobic groups can be:

trimethylsiloxyl groups, which are notably obtained by treatment of pyrogenic silica in the presence of hexamethyldisilazane. Silicas treated in this way are called “Silica silylate” according to the CTFA (6th edition, 1995). They are for example marketed under the references “AEROSIL R812®” by the company Degussa, “CAB-O-SIL TS-530®” by the company Cabot; or “VM-2270 AEROGEL FINE PARTICLES” by Dow.

dimethylsilyloxyl or polydimethylsiloxane groups, which are notably obtained by treatment of pyrogenic silica in the presence of polydimethylsiloxane or dimethyldichlorosilane. Silicas treated in this way are called “Silica dimethyl silylate” according to the CTFA (6th edition, 1995). They are for example marketed under the references; “AEROSIL R972®”, “AEROSIL R974®” by the company Degussa, “CAB—O-SIL TS-610®”, “CAB-O-SIL TS-720®” by the company Cabot.

Also, silica aerogel particles can be added to compositions of the present invention, if desired. Silica aerogels are porous materials obtained by replacing (by drying) the liquid component of a silica gel with air. They are generally synthesized via a sol-gel process in a liquid medium and then dried, usually by extraction with a supercritical fluid, the one most commonly used being supercritical CO2. This type of drying makes it possible to avoid shrinkage of the pores and of the material.

Hydrophobic silica aerogel particles which may be used in the present invention can have a specific surface area per unit of mass (SM) ranging from 500 to 1500 m2/g, preferably from 600 to 1200 m2/g and better still from 600 to 800 m2/g, and a size expressed as the volume-average diameter (D[0.5]) ranging from 1 to 1500 μm, better still from 1 to 1000 μm, preferably from 1 to 100 μm, in particular from 1 to 30 μm, more preferably from 5 to 25 μm, better still from 5 to 20 μm and even better still from 5 to 15 μm.

According to one embodiment, the hydrophobic silica aerogel particles which may be used in the present invention have a size, expressed as volume-average diameter (D[0.5]), ranging from 1 to 30 μm, preferably from 5 to 25 μm, better still from 5 to 20 μm and even better still from 5 to 15 μm.

The sizes of the silica aerogel particles may be measured by static light scattering using a commercial particle size analyser such as the MasterSizer 2000 machine from Malvern. The data are processed on the basis of the Mie scattering theory. This theory, which is exact for isotropic particles, makes it possible to determine, in the case of non-spherical particles, an “effective” particle diameter. According to one advantageous embodiment, the hydrophobic silica aerogel particles which may be used in the present invention have a specific surface area per unit of mass (SM) ranging from 600 to 800 m2/g and a size expressed as the volume-average diameter (D[0.5]) ranging from 5 to 20 μm and even better still from 5 to 15 μm.

The silica aerogel particles which may be used in the present invention may advantageously have a tapped density ρ ranging from 0.02 g/cm3 to 0.10 g/cm3, preferably from 0.03 g/cm3 to 0.08 g/cm3 and preferably from 0.05 g/cm3 to 0.08 g/cm3.

According to one preferred embodiment, the hydrophobic silica aerogel particles which may be used in the present invention have a specific surface area per unit of volume SV ranging from 5 to 60 m2/cm3, preferably from 10 to 50 m2/cm3 and better still from 15 to 40 m2/cm3. The specific surface area per unit of volume is given by the relationship: SV=SM×ρ, where ρ is the tapped density, expressed in g/cm3, and SM is the specific surface area per unit of mass, expressed in m2/g, as defined above.

Preferably, the hydrophobic silica aerogel particles which may be used according to the invention have an oil-absorbing capacity, measured at the wet point, ranging from 5 to 18 ml/g, preferably from 6 to 15 ml/g and better still from 8 to 12 ml/g. The absorbing capacity measured at the wet point, noted Wp, corresponds to the amount of oil that needs to be added to 100 g of particles in order to obtain a homogeneous paste. It is measured according to the “wet point” method or the method for determining the oil uptake of a powder described in standard NF T 30-022. It corresponds to the amount of oil adsorbed onto the available surface of the powder and/or absorbed by the powder by measurement of the wet point, described below: An amount m=2 g of powder is placed on a glass plate, and the oil (isononyl isononanoate) is then added dropwise. After addition of 4 to 5 drops of oil to the powder, mixing is carried out using a spatula, and addition of oil is continued until conglomerates of oil and powder have formed. From this point, the oil is added at the rate of one drop at a time and the mixture is subsequently triturated with the spatula. The addition of oil is stopped when a firm, smooth paste is obtained. This paste must be able to be spread on the glass plate without cracking or forming lumps. The volume Vs (expressed in ml) of oil used is then noted. The oil uptake corresponds to the ratio Vs/m.

The aerogels which may be used according to the present invention are hydrophobic silica aerogels, preferably of silyl silica (INCI name: silica silylate). The term “hydrophobic silica” is understood to mean any silica of which the surface is treated with silylating agents, for example with halogenated silanes, such as alkylchlorosilanes, siloxanes, in particular dimethylsiloxanes, such as hexamethyldisiloxane, or silazanes, so as to functionalize the OH groups with silyl Si—Rn groups, for example trimethylsilyl groups. Silica silylate is particulate hydrophobic silica having surface trialkylsiloxyl groups.

The non-whitening fillers may be coated or uncoated.

In certain embodiments the non-whitening fillers have an average particle size in a range from about 1 micron to about 100 microns, such as from about 1 micron to about 10 microns.

Other fillers that may be included in the composition (explicitly not included among non-whitening fillers) include talc, boron nitride, micas (such as synthetic mica/synthetic fluorphlogopite and natural mica), bismuth oxychloride, and perlite.

While in certain embodiments of the invention the composition may include such fillers, according to certain notable embodiments of the invention, the compositions are substantially free of all of these: micas, talc, perlite, bismuth oxychloride and boron nitride. In certain other embodiments, the talc and mica that are restricted in the composition are limited to those materials that are free from multi-layer coatings that provide interference pigment effects. In other words, in these embodiments, the compositions may include talc or micas that are interference (color) pigments, but are otherwise substantially free of mica, talc, perlite, bismuth oxychloride, and boron nitride (fillers).

Examples of boron nitride include SOFTOUCH BORON NITRIDE POWDER CC6058, commercially available from Momentive Performance Materials, Waterford, New York. Examples of perlite (non-crystalline volcanic glass) include those with a typical composition of 70-75% silicon dioxide, 12-15% aluminum oxide, 3-5% potassium oxide, 3-4% sodium oxide and traces of iron oxide, magnesium oxide and calcium oxide.

The total concentration by weight of fillers are present in low amounts in the compositions of the present invention. The total concentration of fillers in the composition are less than about 4%, such as less than about 3%, such as from about 0.5%, 1%, or 1.5% to about 2%, 3% or 4% by weight.

Compositions of the present invention include one or more inorganic pigments such as one or more color pigments and/or titanium dioxide.

The color pigment provides hiding powder to aid in concealing skin imperfections while imparting some additional visible color. Suitable color pigments include any of various inorganic pigments such as iron oxides, ultramarine blue pigments, manganese violet, ferric ferrocyanide and chromium green pigments, carbon black, and the like. Iron oxide is particularly notable. The color pigments may be coated or uncoated, however in certain embodiments of the invention, the color pigments are uncoated.

Any of various cosmetic grades of color pigments are suitable for use in compositions of the present invention. In certain embodiments the color pigments have an average particle size in a range from about 1 micron to about 100 microns, such as from about 1 micron to about 10 microns.

The concentration of the color pigments in the composition may range from about 0.01%, 0.05%, or 0.5% to about 1%, 2%, or 30% by weight, including all ranges and subranges therebetween. In certain embodiments of the invention, the color pigments are present in the composition in a concentration by weight of at least about 5%, such as at least about 10%, such as from about 5%, 10% or 12% to about 15%, 20% or 30% by weight.

Furthermore, compositions of the present invention may further include an opacifying pigment such as titanium dioxide. The opacifying pigment provides hiding powder, but is generally used in concentrations to limit whitening effects on the skin. The opacifying pigment, preferably titanium dioxide may be present in a concentration of up to about 30% by weight in the composition, such as from about 0%, 1% or 2% to about 2%, 4%, 5%, 6%, 8%, 10%, 15%, 20%, 25% or 30%. In certain preferred embodiments, the compositions include a color pigment and titanium dioxide.

In certain embodiments the total concentration of pigments (color pigments and opacifying pigments is at least about 25%, such as from about 25% or 30% to about 30% or 35% by weight. In certain other embodiments, the ratio of color pigments to opacifying pigments is at least about 1:5, such as at least about 1:1. In certain embodiments, the ratio of color pigments to opacifying pigments is from about 1:1 to about 100:1.

As described above, compositions of the present invention include one or more lipids, such as at least one oil. By “lipid,” it is meant any of various fatty substances insoluble in water and including, for example, hydrocarbon based fatty substances or silicone-based fatty substances. These may be liquid or solid at room temperature and may be volatile or non-volatile.

By “oil,” it is meant compounds having a melting point of less than about 15° C., such as less than about 10° C., and generally insoluble in water and includes a hydrophobic moiety, such as one meeting one or more of the following three criteria: (a) has a carbon chain of at least six carbons in which none of the six carbons is a carbonyl carbon or has a hydrophilic moiety (defined below) bonded directly to it; (b) has two or more alkyl siloxy groups; or (c) has two or more oxypropylene groups in sequence. The hydrophobic moiety may include linear, cyclic, aromatic, saturated or unsaturated groups. The hydrophobic compound is in certain embodiments not amphiphilic and, as such, in this embodiment does not include hydrophilic moieties, such as anionic, cationic, zwitterionic, or nonionic groups, that are polar, including sulfate, sulfonate, carboxylate, phosphate, phosphonate, ammonium, including mono-, di-, and trialkylammonium species, pyridinium, imidazolinium, amidinium, poly(ethyleneiminium), ammonioalkylsulfonate, ammonioalkylcarboxylate, amphoacetate, and poly(ethyleneoxy)sulfonyl moieties. In certain embodiments, the oil does not include hydroxyl moieties. The oil may have a weight average molecular weight less than about 1000 daltons.

Suitable examples of compounds of oils include vegetable oils (glyceryl esters of fatty acids, monoglycerides, diglycerides, triglycerides) and fatty esters. Specific non-limiting examples include, without limitation, esters such as isopropyl palmitate, isopropyl myristate, isononyl isonanoate C12-C15 alkyl benzoates, caprylic/capric triglycerides, ethylhexyl palmitate, silicone oils (such as dimethicone and cyclopentasiloxane), pentaerythritol tetraoctanoate and mineral oil. Other examples of oils include liquid organic ultraviolet filter commonly used for example as UV-absorbing sunscreens such as octocrylene, octyl salicylate, octyl methoxyxcinnamate, among others.

Suitable oils include volatile and/or non-volatile oils. Such oils can be any acceptable oil including but not limited to silicone oils and/or hydrocarbon oils.

According to certain embodiments, the compositions of the present invention may include one or more volatile silicone oils. Examples of such volatile silicone oils include linear or cyclic silicone oils having a viscosity at room temperature less than or equal to 6cSt and having from 2 to 7 silicon atoms, these silicones being optionally substituted with alkyl or alkoxy groups of 1 to 10 carbon atoms. Specific oils that may be used in the invention include octamethyltetrasiloxane, decamethylcyclopentasiloxane, dodecamethylcyclohexasiloxane, heptamethyloctyltrisiloxane, hexamethyldisiloxane, decamethyltetrasiloxane, dodecamethylpentasiloxane and their mixtures. Other volatile oils which may be used include KF 96A of 6 cSt viscosity, a commercial product from Shin Etsu having a flash point of 94° C.

The concentration of oils in the composition is preferably at least about 25% such as from about 25%, 30% or 35% by weight to about 35%, 40% or 50% by weight, including all combinations of such ranges, relative to the total weight of the composition.

Compositions of the present invention may include other lipids such as at least one wax. As used herein, “wax” is intended to mean a lipophilic fatty compound that is solid at room temperature (about 25° C.) and atmospheric pressure (760 mm Hg, i.e., 105 Pa), which undergoes a reversible solid/liquid change of state and which has a melting point of greater than 30° C., and in some embodiments, greater than about 55° C. up to about 120° C. or even as high as about 200° C. In certain embodiments the wax has a weight average molecular weight of less than about 2000 daltons.

The wax may change from the solid to the liquid state reversibly, and/or may have a hardness of more than 0.5 MPa at ambient temperature, and an anisotropic crystalline organization in the solid state.

The term wax may include waxes of animal origin, waxes of plant origin, waxes of mineral origin and waxes of synthetic origin. Examples of waxes of animal origin include beeswaxes, lanolin waxes and Chinese insect waxes. Examples of waxes of plant origin include rice waxes, carnauba wax, candelilla wax, ouricurry wax, cork fiber waxes, sugar cane waxes, Japan waxes, sumach wax and cotton wax. Examples of waxes of mineral origin include paraffins, microcrystalline waxes, montan waxes and ozokerites. Examples of waxes of synthetic origin include polyolefin waxes, e.g., polyethylene waxes (linear, low molecular weight polyethylene waxes), waxes obtained by Fischer-Tropsch synthesis, waxy copolymers and their esters, and silicone and fluoro waxes.

The term wax may further include high melting point hydrogenated oils of animal or plant origin. Examples include hydrogenated jojoba waxes and hydrogenated oils which are obtained by catalytic hydrogenation of fats composed of a C8-C32 linear or nonlinear fatty chain, hydrogenated sunflower oil, hydrogenated castor oil, hydrogenated copra oil, hydrogenated lanolin and hydrogenated palm oils.

While in certain embodiments, compositions of the present invention include from about 1% to about 10% wax. However, in certain other embodiments, compositions of the present invention have only limited amounts of wax, such as less than about 5% by weight, such as from about 1% to about 4% by weight.

In certain embodiments of the invention, the composition may include other lipids which may not fall into the definition of wax or oils, such as fatty materials that are semi-solid, pastes or paste-like at room temperature (“paste-like compound”), e.g., have a melting temperature in a range from about 15° C. to about 40° C. Suitable examples include shea butter, hydrogenated butters, and any of various partial esters of diglycerin with medium chain fatty acids, such as Bis-Diglyceryl Polyacyladipate-2, commercially available as SOFTISAN 649 from Cremer Oleo of Hamburg, Germany; and ethylhexyl hydroxystearate such as WICKENOL 171 available from Alzo International of Sayerville, NJ. In certain embodiments, the compositions of the invention include from about 5% or 7% or 8% to about 12% or 15% of the paste-like compound.

Examples of other suitable lipids include hydroxylated fatty acid esters. The hydroxylated fatty acid esters may have a high melting point. By “high melting point hydroxylated fatty acid ester” it is meant a hydroxylated fatty acid ester having a melting point of at least 40° C. In certain embodiments the hydroxylated fatty acid ester has a melting point of at least about 50° C., such as at least about 60° C., such as at least about 80° C.

According to certain embodiments the high melting point hydroxylated fatty acid ester is a triester of a fatty acid and glycerol (fatty acid glycerol triester). According to certain embodiments the high melting point hydroxylated fatty acid ester has at least one C12 or greater carbon chain. In certain other embodiments, it has at least two hydroxyl groups. In certain other embodiments, the high melting point hydroxylated fatty acid ester is trihydroxystearin (THS).

One notable high melting point hydroxylated fatty acid ester suitable for use in the composition is the hydroxystearin THIXCIN R, commercially available from Elementis Specialties, East Windsor, New Jersey.

The concentration of the high melting point hydroxylated fatty acid ester in the composition may range from about 0.1% to about 5% by weight, such as from about 0.1% to about 1%, such as from about 0.25% to about 1% by weight in the composition.

The total concentration of lipids in the composition may be from about 10%, 15%, 20%, 25%, or 35% by weight to about 40%, 55% or 60% by weight, including all combinations of such ranges, relative to the total weight of the composition.

Compositions of the present invention may optionally include other functional ingredients such as those that can be readily dissolved, dispersed or suspended in the composition. These may include

at least one unsaturated aliphatic tackifier having a weight average molecular weight of less than about 10,000 daltons, such as less than about 5000 daltons, such as from about 1500 to about 4000. The aliphatic tackifier generally provides enhanced skin adhesion suitable for use in spot treatment (e.g, “concealer” formulas). By “tackifier,” it is meant a material that enhances surface adhesion and may have a glass transition temperature less than about 0° C. (such as measured by DTA or DSC). By “aliphatic” is meant a hydrocarbon based compounds or material that are straight, branched, or cyclic, but are free of aromatic ring structures. According to certain notable embodiments, the aliphatic tackifier is unsaturated, such as a polybutene. Polybutene tackifiers may be made by polymerization of C4 olefins (e.g, primarily isobutene) and may be, for example, free-flowing, sticky with a honey-like consistency, or very tacky, semi-solid materials. Such synthetic hydrocarbon tackifiers may have a viscosity between about 1000 centistokes (cSt) and 5000 cSt when measured at 100° C. One notable aliphatic tackifier is INDOPOL H 1500, commercially available from Ineos Capital of London, England.

According to certain embodiments, the concentration of the unsaturated aliphatic tackifier in the composition is at least about 10% such as from about 10%, 15%, 20% by weight, to about 20% or 30% by weight in the composition.

Other functional ingredients may include an activator for the optional clay. A suitable activator is an organic carbonate ester. According to certain embodiments of the invention, the organic carbonate ester is a C1-C4 alkylene carbonate such as ethylene carbonate or propylene carbonate and most notably, propylene carbonate. The organic carbonate ester may be, as noted above added via the same raw material as the swellable clay. Alternatively, it may be added separately.

Other functional ingredients that may be included in the composition include a film forming polymer. Film-forming polymers are polymers or resins that leave a film (e.g., a continuous film) on the substrate to which it is applied, for example, after a solvent accompanying the film former has evaporated, absorbed into and/or dissipated on or from the substrate. According to certain embodiments, the film-forming polymer forms a conformal coating and/or can picked up or scraped off with a razorblade to be removed as a free standing film when tested in a drawdown test, such as one performed by putting 5 to 10 grams of material on the center of a Leneta card stock (Black and White Opacity card Chart 2812 available from BYK Additives and Instruments of Geretsried, Germany) and using a 3 mil Drawdown Birdbar (also from Byk), spreading the material for across the sheet (8 in by 3 in) and allowing it to dry overnight.

The film-forming polymer may have one or more of acrylic, urethane, ester, vinyl, silicone or other functionality. In certain embodiments the film-forming polymer is a polysaccharide or protein-based polymer. According to certain embodiments, the film-forming polymer is a vinyl polymer, such as VP/hexadecane copolymer, available as GANEX V216 from Ashland Global Specialty Chemicals Inc of Wilmngton, Delaware. If present, the film-forming polymer may be present in a concentration from about 0.25%, 0.5% or 1% to about 1%, 2%, 5%, or 10% by weight in the composition.

Other functional ingredients that may be included in the composition include other particulate materials (organic, silicone-based); oligomers or polymers such as for thickening/rheology modifying, tackifying or film-forming; preservatives; dyes, fragrances; antioxidants; sunscreens; skin actives including vitamins and the like. A person skilled in the art will take care to select the optional additional ingredients and/or the amount thereof such that the advantageous properties of the composition according to the invention are not, or are not substantially, adversely affected by the envisaged addition.

These substances may be selected variously by the person skilled in the art in order to prepare a composition which has the desired properties, for example, consistency or texture.

Other functional ingredients that may be included in the composition include surfactants or emulsifies. While in certain embodiments, compositions of the present invention include a surfactant or emulsifier, in certain other notable embodiments, the composition is substantially free of surfactants and emulsifiers.

The other functional ingredients may be present in the composition in concentrations up to about 20%, such as from about 0%, 2%, or 5% to about 10%, 15%, or 20%, including all ranges and subranges therebetween.

According to certain embodiments, compositions of the present invention include (1) from about 25% to about 70% by weight of one or more lipids, wherein the one or more lipids include (a) at least one oil, (b) a high melting point hydroxylated fatty acid ester; (c) at least one wax and (d) a paste-like compound; (2) one or more inorganic particulates, wherein the one or more inorganic particulates include (a) at least one filler, present in a concentration by weight of from about 0.5% by weight to about 4% with respect to the entire composition, wherein the at least one filler is selected from a group consisting of silica aerogel, borosilicate, swellable clay, and combinations thereof; (b) one or more pigments, present in a concentration by weight of at least about 25% by weight with respect to the entire composition; wherein the one or more pigments include a pigment selected from color pigments, titanium dioxide, and combinations thereof; and (3) from about 10% by weight to about 30% by weight of an unsaturated aliphatic tackifier having a weight average molecular weight of less than about 10,000 daltons; wherein the composition is substantially free of water, boron nitride, talc, mica, bismuth oxychloride and perlite. The paste-like compound may be in a concentration by weight from about 5% to about 12% of the entire composition.

The compositions may be made with various orders of addition. For example, oils, waxes, and solid ingredients (non-whitening fillers and color pigments and unsaturated aliphatic tackifier) may be melted to create a uniform liquid mixture. After allowing to cool to approximately 60° C., trihydroxystearin and pasty compounds can be then mixed in with high shear until homogeneous. The remaining ingredients, the swellable clay (BENTONE GEL GTCC) and preservatives/actives and can then added to the container with stirring at approximately 60° C. until a homogeneous fluid composition is obtained. The composition is then allowed to cool to room temperature. Alternatively, swellable clay may be added with the oils, wax and fillers.

According to preferred embodiments of the present invention, methods of treating, caring for and/or making up a keratinous material by applying compositions of the present invention to the keratinous material in an amount sufficient to treat, care for and/or make up the keratinous material are provided. According to yet other preferred embodiments, methods of enhancing the appearance of a keratinous material by applying compositions of the present invention to the keratinous material in an amount sufficient to enhance the appearance of the keratinous material are provided.

In accordance with the preceding preferred embodiments, the compositions of the present invention are applied topically to the desired area of the keratinous material in an amount sufficient to treat, care for and/or make up the keratinous material, to spot-treat, cover or hide spots, pigmentation discontinuities or defects associated with keratinous material, or to enhance the appearance of keratinous material. The compositions may be applied to the desired area as needed, preferably once daily, and then preferably allowed to dry before subjecting to contact such as with clothing or other objects. Preferably, the composition is allowed to dry for about 4 minutes or less, more preferably for about 2 minutes or less.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective measurements. The following examples are intended to illustrate the invention without limiting the scope as a result. The percentages are given on a weight basis.

### Example 1—Inventive Compositions

Compositions consistent with the ingredients and concentrations below were prepared:

| | | INVENTIVE | INVENTIVE | INVENTIVE |
|---|---|---|---|---|
| | | EX. 1 | EX. 2 | EX. 3 |
| FUNCTION/TYPE | INGREDIENT | CONCENTRATION BY WT. | | |
| Oils | Caprylic/capric | 33% | 33% | 33% |
| | triglyceride | | | |
| Waxes | Various | 3% | 3% | 4% |
| High melting point | THS | 0.6% | 0.6% | 0.5% |
| hydroxylated fatty | | | | |
| acid ester | | | | |
| Paste-Like | Various | 11% | 11% | 10% |
| Compounds | | | | |
| Non-whitening | Hectorite, silica | 3% | 3% | 1% |
| filler | aerogel, calcium | | | |
| | aluminum | | | |
| | borosilicate | | | |
| Opacifying | Titanium dioxide | 27% | 0% | 27% |
| pigment | | | | |
| Color pigment | Iron oxides | 3% | 30% | 3% |
| Other filler | Boron nitride | | | 1% |
| Unsaturated | Polybutene | 13% | 13% | 14% |
| aliphatic tackifier | | | | |
| Film-Forming | Vinyl copolymer | 5% | 5% | 5% |
| Polymer | | | | |
| Other ingredients | Preservatives, skin | Q.S. | Q.S. | Q.S. |
| | actives, etc. | | | |

For inventive examples 1, 2 and 3, the oils, waxes, and solid ingredients (non-whitening fillers and color pigments and unsaturated aliphatic tackifier) were melted to create a uniform liquid mixture. After allowing to cool to approximately 60° C., trihydroxystearin and pasty compounds were mixed in with high shear until homogeneous. The remaining ingredient, the swellable clay (BENTONE GEL GTCC) and preservatives/actives and were then added to the container with stirring at approximately 60° C. until a homogeneous fluid composition was obtained. The composition was then allowed to cool to room temperature. The consistency was such that compositions could be filled into tubes that could be squeezed to release the composition. Inventive examples 1 and 2 were free of whitening fillers such as mica, talc, perlite, bismuth oxychloride, and boron nitride. The compositions were applied to skin and had a surprisingly pleasant feel upon application, good tack, appropriate shine, high skin coverage, good break/texture. Furthermore, the inventive examples including inventive example 2, which had a very high concentration of color pigments, still showed high color intensity without providing a whitish cast. Inventive example 3 while still showing some whitish cast (particularly as titanium dioxide in the composition is increasingly substituted with color pigments), showed a dramatic color intensity boost as compared with comparative examples below.

### Example 2—Comparative Compositions

Comparative compositions with the ingredients and concentrations below was also prepared, as shown in the tables below.

| | | COMP | COMP | COMP | COMP |
|---|---|---|---|---|---|
| | | EX. 1 | EX. 2 | EX. 3 | EX. 4 |
| FUNCTION/TYPE | INGREDIENT | CONCENTRATION BY WT. % | | | |
| Oils | Caprylic/capric | 27% | 27% | 32% | 31% |
| | triglyceride | | | | |
| Waxes | Various | 3% | 3% | 3% | 4% |
| High melting point | THS | 0.5% | 0.5% | 0.5% | 0.5% |
| hydroxylated fatty | | | | | |
| acid ester | | | | | |
| Paste-Like | Various | 6% | 9% | 9% | 10% |
| Compounds | | | | | |
| Non-whitening | Hectorite, silica | 1% | 1% | 2% | 1% |
| filler | aerogel, | | | | |
| | calcium | | | | |
| | aluminum | | | | |
| | borosilicate | | | | |
| Opacifying | Titanium | 26% | 26% | 26% | 24% |
| pigment | dioxide | | | | |
| Color pigment | Iron oxides | 4% | 4% | 4% | 3% |
| Other Fillers | Perlite, BiOCl, | 10% | 10% | 3% | 6% |
| | Mica, Boron | | | | |
| | nitride | | | | |
| Unsaturated | polybutene | 16% | 12% | 14% | 14% |
| aliphatic tackifier | | | | | |
| Film-Forming | Vinyl polymer | 5% | 5% | 5% | 5% |
| Polymer | | | | | |
| Other ingredients | Preservatives, | Q.S. | Q.S. | Q.S. | Q.S. |
| | skin actives, | | | | |

An additional comparative composition with the ingredients and concentrations below was also prepared.

| | | COMP |
|---|---|---|
| | | EX. 5 |
| FUNCTION/TYPE | INGREDIENT | CONCENTRATION BY WT. % |
| Oils | Caprylic/capric | 33% |
| | triglyceride | |
| Waxes | Various | 4% |
| High melting point | THS | 0.5% |
| hydroxylated fatty | | |
| acid ester | | |
| Paste-Like | Various | 10% |
| Compounds | | |
| Non-whitening | Hectorite, silica | 1% |
| filler | aerogel, | |
| | calcium | |
| | aluminum | |
| | borosilicate | |
| Opacifying | Titanium | 24% |
| pigment | dioxide | |
| Color pigment | Iron oxides | 3% |
| Other Fillers | Perlite, BiOCl, | 4% |
| | Mica, Boron | |
| | nitride | |
| Unsaturated | polybutene | 14% |
| aliphatic tackifier | | |
| Film-Forming | Vinyl polymer | 5% |
| Polymer | | |
| Other ingredients | Preservatives, | Q.S. |
| | skin actives, | |
| | etc. | |

For Comparative Examples 1-5, a similar process to making the inventive examples was followed.

The comparative examples showed surprisingly inferior properties to the inventive examples. Comparative Example 1 showed, caky-ness, high opacity, and dullness. Comparative Example 2 showed high opacity, dullness. and slow break on spreading. Comparative Example 3 showed slow break on spreading and dragginess. Comparative Example 4 showed acceptable tactile properties, but was when additional color pigment was substituted for opacifying pigment, the sample provided poor color intensity, i.e. an unwelcome whitish cast was visible on the skin upon using the product as a concealer. Comparative Example 5 also demonstrated a whitish cast that was made considerably worse when the same formula had color pigment substituted for all of the titanium dioxide (27% iron oxide, 0% titanium dioxide).

The foregoing description illustrates and describes the disclosure. Additionally, the disclosure shows and describes only the preferred embodiments but, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. accordingly, the description is not intended to limit the invention to the form disclosed herein. Also, it is intended to the appended claims to construed to include alternative embodiments.

1. A composition, comprising:
at least about 25% by weight of one or more oils;
from 0.5% by weight to about 4% by weight of one or more fillers, wherein the one or more fillers comprise silica aerogel, borosilicate, swellable clay, and combinations thereof; and
at least about 25% by weight of one or more pigments, wherein the composition is substantially free of water.

2. The composition of claim 1 wherein the composition is substantially free of fillers selected from the group consisting of micas, perlite, talc, bismuth oxychloride and boron nitride, and combinations thereof.

3. The composition of claim 1 wherein the composition has a combined concentration of lipids and inorganic particulates that is at least about 60% by weight.

4. The composition of claim 1 further comprising an unsaturated aliphatic tackifier having a weight average molecular weight of less than about 10,000 daltons.

5. The composition of claim 4 wherein the unsaturated aliphatic tackifier is polybutene.

6. The composition of claim 4 wherein the unsaturated aliphatic tackifier is present in a concentration from about 10% by weight to about 30% by weight in the composition.

7. The composition of claim 1 further comprising a high melting point hydroxylated fatty acid ester.

8. The composition of claim 7, wherein the high melting point hydroxylated fatty acid ester is trihydroxystearin.

9. The composition of claim 1, wherein the composition comprises from about 25% by weight to about 70% by weight of lipids and wherein the composition comprises from about from about 15% by weight to about 60% by weight of inorganic particulates.

10. The composition of claim 9, wherein the lipids and inorganic particulates are present in a weight ratio of lipid to inorganic particulate that is from about 1:2 to about 2:1.

11. The composition of claim 1, wherein the composition further comprises wax in a concentration by weight of wax that is less than about 5%.

12. The composition of claim 1 wherein the one or more pigments comprise at least one color pigment, and wherein the at least one color pigment is present in the composition in a concentration by weight of at least about 5%.

13. A method of concealing imperfections on a keratinous substrate comprising topically applying the composition of claim 1 to selected imperfections on the keratinous substrate.
